(19) United States
(12) Patent Application Publication
(10) Pub. No.: US 2008/0234340 A1
(43) Pub. Date: Sep. 25, 2008

(54) Synthesis and characterization of polymorph form II of 4-(2-(4,4-dimethyl-2-oxooxazolidin-3-YL)thiazol-4-YL)benzonitrile

(75) Inventors: Mahmoud Mirmehrabi, Laval (CA); Yuping Niu, River Vale, NJ (US); Abdolsamad Tadayon, Montreal (CA); Subodh Deshmukh, White Plains, NY (US); Mannching Sherry Ku, Thiells, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/075,228

(22) Filed: Mar. 10, 2008

Related U.S. Application Data
(60) Provisional application No. 60/906,094, filed on Mar. 9, 2007.

(51) Int. Cl. A61K 31/427; C07D 413/02; A61P 35/00; A61P 5/24

(52) U.S. Cl. 514/376; 548/229

(57) ABSTRACT

A polymorph Form II of 4-(2-(4,4-dimethyl-2-oxooxazolidin-3-yl)thiazol-4-yl)benzonitrile and methods of preparing Form II are described. Also provided are methods of contraception, treating or preventing fibroids, uterine leiomyomata, endometriosis, dysfunctional bleeding, polycystic ovary syndrome, and hormone-dependent carcinomas, providing hormone replacement therapy, stimulating food intake, and synchronizing estrus including using polymorph Form II of 4-(2-(4,4-dimethyl-2-oxooxazolidin-3-yl)thiazol-4-yl)benzonitrile.

Further provided are methods for preparing polymorph Form I of 4-(2-(4,4-dimethyl-2-oxooxazolidin-3-yl)thiazol-4-yl)benzonitrile from polymorph Form II of 4-(2-(4,4-dimethyl-2-oxooxazolidin-3-yl)thiazol-4-yl)benzonitrile.

This application claims the benefit of U.S. Provisional Application No. 60/906,094, filed Mar. 9, 2007, the entire disclosure of which is incorporated herein by reference.

### BACKGROUND OF THE INVENTION

This invention relates to polymorphs of modulators of the progesterone receptor, their preparation and utility.

Intracellular receptors (IR) form a class of structurally related gene regulators known as “ligand dependent transcription factors” (Mangelsdorf, D. J. etc. Cell, 83, 835, 1995). The steroid receptor family is a subset of the IR family, including the progesterone receptor (PR), estrogen receptor (ER), androgen receptor (AR), glucocorticoid receptor (GR), and mineralocorticoid receptor (MR).

The natural hormone, or ligand, for the PR is the steroid progesterone, but synthetic compounds, such as medroxyprogesterone acetate or levonorgestrel, can which also serve as PR ligands. Once a ligand is present in the fluid surrounding a cell, it passes through the membrane via passive diffusion, and binds to the IR to create a receptor/ligand complex. This complex binds to specific gene promoters present in the cell's DNA. Once bound to the DNA the complex modulates the production of mRNA and the protein encoded by that gene.

A compound that binds to an IR and mimics the action of the natural hormone is termed an agonist, while a compound which inhibits the effect of the hormone is an antagonist. Thus, both PR agonists and antagonists can modulate the activity of progesterone receptors; a PR antagonist inhibits PR activation and a PR agonist mimics the activity of progesterone.

PR agonists (natural and synthetic) are known to play an important role in the health of women. PR agonists are used in birth control formulations, either alone or in the presence of an ER agonist. Progestin therapy has been used to increase appetite.

PR antagonists can also be used in contraception (Ulmann, et al., Ann. N.Y. Acad. Sci., 261, 248, 1995; Kekkonen, et al, Fertility and Sterility, 60, 610, 1993; U.S. Pat. No. 5,719,136); for the treatment of hormone dependent breast cancers (Horwitz, et al, Horm. Cancer, 283, 1996, pub: Birkhaeuser, Boston, Mass., ed. Vedeckis), uterine and ovarian cancers, non-malignant chronic conditions such as uterine fibroids (Murphy, et al, J. Clin. Endo. Metab., 76, 513, 1993) and endometriosis (Kettel, et al., Fertility and Sterility, 56, 402, 1991), and hormone dependent prostate cancer (Michna, et al, Ann. N.Y. Acad. Sci., 761, 224, 1995); and for hormone replacement therapy (U.S. Pat. No. 5,719,136).

As drug formulations possessing high bioavailability and long-term stability are highly desirable, there is an ongoing need for crystalline drug molecules with such characteristics, including alternate forms of progesterone receptor modulators such as 4-(2-(4,4-dimethyl-2-oxooxazolidin-3-yl)thiazol-4-yl)benzonitrile.

### SUMMARY OF THE INVENTION

In one aspect, polymorph Form II of 4-(2-(4,4-dimethyl-2-oxooxazolidin-3-yl)thiazol-4-yl)benzonitrile is described.

In another aspect, methods of preparing polymorph Form II of 4-(2-(4,4-dimethyl-2-oxooxazolidin-3-yl)thiazol-4-yl)benzonitrile are described.

In a further aspect, methods of contraception, treating or preventing fibroids, uterine leiomyomata, endometriosis, dysfunctional bleeding, polycystic ovary syndrome, and hormone-dependent carcinomas, providing hormone replacement therapy, stimulating food intake, and synchronizing estrus are described and include administering polymorph Form II of 4-(2-(4,4-dimethyl-2-oxooxazolidin-3-yl)thiazol-4-yl)benzonitrile to a mammal in need thereof.

In yet another aspect, methods for preparing polymorph Form I of 4-(2-(4,4-dimethyl-2-oxooxazolidin-3-yl)thiazol-4-yl)benzonitrile from polymorph Form II of 4-(2-(4,4-dimethyl-2-oxooxazolidin-3-yl)thiazol-4-yl)benzonitrile are described.

Other aspects and advantages of the present invention are described further in the following detailed description of the preferred embodiments thereof.

### BRIEF DESCRIPTION OF THE DRAWINGS

A complete understanding of the present invention may be obtained by reference to the accompanying drawings when considered in conjunction with the following detailed description, in which:

FIG. 1 provides the X-ray diffraction (XRD) pattern for a sample of polymorph Form II of 4-(2-(4,4-dimethyl-2-oxooxazolidin-3-yl)thiazol-4-yl)benzonitrile.

FIG. 2 provides a first differential scanning calorimetry (DSC) thermograph for a sample of polymorph Form II of 4-(2-(4,4-dimethyl-2-oxooxazolidin-3-yl)thiazol-4-yl)benzonitrile.

FIG. 3 provides a second differential scanning calorimetry (DSC) graph for a sample of polymorph Form II of 4-(2-(4,4-dimethyl-2-oxooxazolidin-3-yl)thiazol-4-yl)benzonitrile.

FIG. 4 provides the solution phase nuclear magnetic resonance (NMR) spectrum for a sample of polymorph Form II of 4-(2-(4,4-dimethyl-2-oxooxazolidin-3-yl)thiazol-4-yl)benzonitrile.

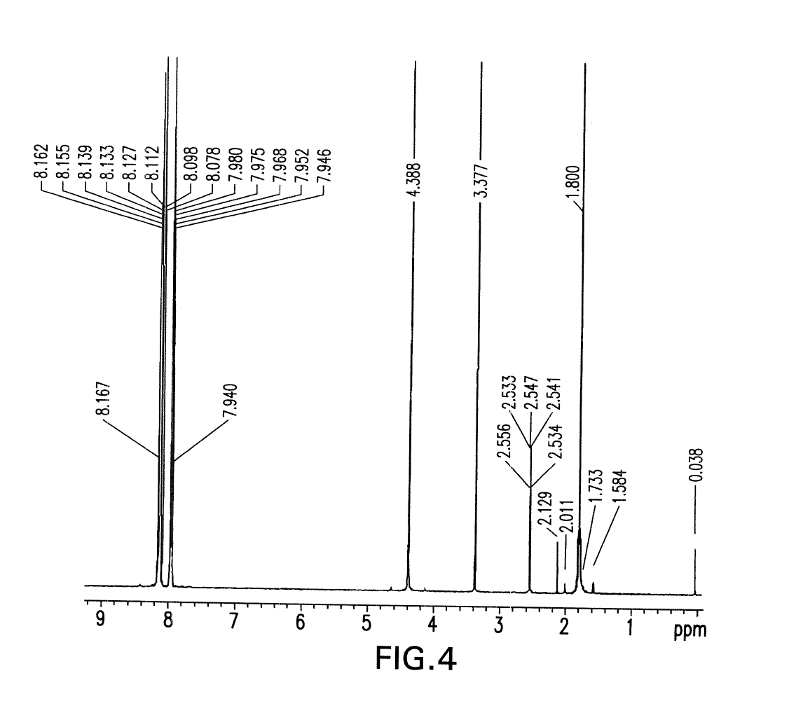

FIG. 5 provides the thermogravimetric analysis (TGA) spectrum for a sample of polymorph Form II of 4-(2-(4,4-dimethyl-2-oxooxazolidin-3-yl)thiazol-4-yl)benzonitrile.

### DETAILED DESCRIPTION OF THE INVENTION

The invention relates to a novel crystalline form of 4-(2-(4,4-dimethyl-2-oxooxazolidin-3-yl)thiazol-4-yl)benzonitrile, denoted herein as Form II. Form II differs from Form I in the structure of the crystal lattice of 4-(2-(4,4-dimethyl-2-oxooxazolidin-3-yl)thiazol-4-yl)benzonitrile Form I, and the two forms give distinctive x-ray powder diffraction (XRD) patterns and differential scanning calorimetry (DSC) thermograms.

As used herein, “Form I” refers to a polymorph of 4-(2-(4,4-dimethyl-2-oxooxazolidin-3-yl)thiazol-4-yl)benzonitrile which can be prepared according to the procedure set forth in U.S. Provisional Patent Application No. 60/837,898, filed Aug. 28, 2006, and U.S. patent application Ser. No. 11/891,748, filed on Aug. 13, 2007, the disclosures of which are hereby incorporated by reference herein in their entirety. References to “Form I” herein include both non-micronized and micronized forms of the same. Micronization of polymorph Form I of 4(2-(4,4-dimethyl-2-oxooxazolidin-3-yl)thiazol-4-yl)benzonitrile is typically accomplished under nitrogen using conventional micronizing techniques, for example with a Trost or jet mill, applied to non-micronized 4-(2-(4,4-dimethyl-2-oxooxazolidin-3-yl)thiazol-4-yl)benzonitrile. Preferably, micronized 4-(2-(4,4-dimethyl-2-oxooxazolidin-3-yl)thiazol-4-yl)benzonitrile Form I has a median particle size less than about 6.4 μm.

Form I of 4-(2-(4,4-dimethyl-2-oxooxazolidin-3-yl)thiazol-4-yl)benzonitrile is characterized by an XRD pattern comprising peaks at 2θ at about 6.0 and 13.1°. The DSC thermograph of Form I of 4-(2-(4,4-dimethyl-2-oxooxazolidin-3-yl)thiazol-4-yl)benzonitrile contains an endothermic peak with a Tₒₙₛₑₜ of about 185° C. Form I further lacks an exothermic peak in the DSC when analyzed by the methods described herein including use of a Q series™ DSC Q1000 DSC instrument.

As used herein, the term “about” means ±10% of the stated value.

The inventors found that polymorph Form II of 4-(2-(4,4-dimethyl-2-oxooxazolidin-3-yl)thiazol-4-yl)benzonitrile is less hygroscopic than Form I. The term “hygroscopic” as used herein refers to the ability of a chemical compound to absorb water/moisture from the air under normal, atmospheric conditions. One of skill in the art would readily be able to measure the hygroscopicity of Form II using techniques known in the art including, without limitation, dynamic vapor sorption (DVS) at relative humidities ranging from 0 to 100%. This decreased hygroscopic property of Form II thereby permits longer shelf life and storage of the solid compound in comparison to Form I.

The inventors also found that Form II of 4-(2-(4,4-dimethyl-2-oxooxazolidin-3-yl)thiazol-4-yl)benzonitrile is more soluble in solvents such as water and hydrophilic non-ionic surfactants than Form I. In one embodiment, the solvent is water. In another embodiment, the solvent is 2% Tween® 80 reagent in water, wherein Tween® 80 is a polyoxyethylene (20) sorbitan monooleate. One of skill in the art would readily be able to measure the solubility of Form II using techniques known in the art. Typically, the solubility was measured by adding Form II to a solvent at room temperature, centrifuging the solution after 24 hours, and assaying the supernatant. This increased solubility is observed when Form II is immersed in water. In fact, Form II is 4 times more soluble in water than Form I.

Characterization of Form II and distinguishing the same from Form I are accomplished using techniques known to those of skill in the art. Specifically, verification that Form II is present can be performed using techniques such as melting point, infrared (IR) spectroscopy, nuclear magnetic resonance (NMR) spectroscopy, mass spectral (MS) analysis, combustion analysis, Raman spectroscopy, elemental analysis, and chromatography including high performance liquid chromatography. Techniques including differential scanning calorimetry (DSC) and X-ray diffraction (XRD) are also useful in distinguishing polymorphs, and specifically Form II from Form I. One or more of the foregoing techniques can be used to identify a polymorphic form of 4-(2-(4,4-dimethyl-2-oxooxazolidin-3-yl)thiazol-4-yl)benzonitrile.

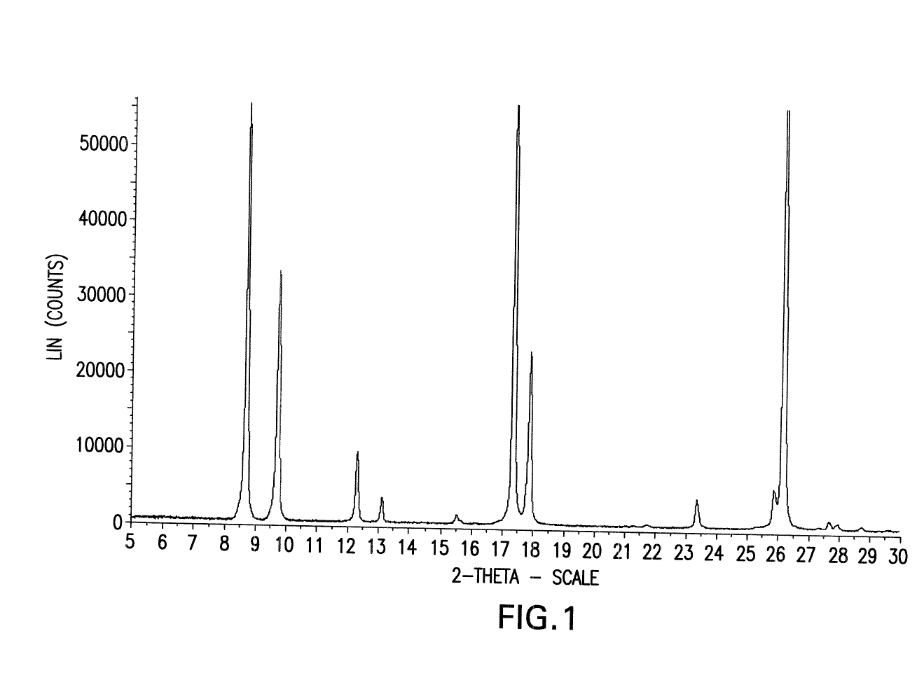

The x-ray powder diffraction pattern for Form II is provided in FIG. 1, and was obtained using X-ray crystallographic techniques known to those of skill in the art. In view of the information provided herein, one of skill in the art would readily be able to determine the conditions required to obtain an XRD pattern of Form II. A variety of XRD instruments are available and include the D8 ADVANCE X-ray powder diffractometer (Bruker) among others. One skilled in the art would recognize that the relative intensities of the peaks in the XRD pattern can vary depending upon the sample preparation technique, the sample mounting procedure and the particular instrument employed. Moreover, instrument variation and other factors can affect the 2-theta values, which can vary by plus or minus about 0.30. All references to XRD peak assignments herein are taken to mean the specified peak ±0.30 (e.g, 9.740 is equivalent to 9.74±0.30) unless otherwise indicated.

Form I and Form II have distinctive characteristic peaks in their x-ray powder diffraction patterns as provided in Table 1. At least one of these peaks, and preferably a majority of these peaks, will be present in the x-ray powder diffraction pattern for a given form.

TABLE 1

| Form I | Form II |
|---|---|
| 6.0 | 8.67 |
| 13.1 | 9.70 |
| | 12.29 |
| | 13.10 |
| | 15.55 |
| | 17.37 |
| | 17.92 |
| | 21.78 |
| | 23.39 |
| | 25.89 |
| | 26.17 |
| | 27.70 |
| | 27.99 |
| | 28.74 |

Preferably, the XRD pattern for Form II exhibits characteristic peaks at 8.67 and 9.70 degrees 2θ. More preferably, the XRD pattern for Form II can comprise peaks at 8.67, 9.70, 17.37, 17.92, and 26.12 degrees 2θ. The XRD pattern for Form II can comprise the following peaks: 8.67, 9.70, 12.29, 13.10, 17.37, 17.92, 23.39, 25.81, and 26.12 degrees 2θ.

In accordance with embodiments of the invention, the XRD patterns for Form I and Form II contain peaks that are specific for each form. The XRD pattern for Form II contains peaks not present in the XRD pattern for Form I, and includes a peak at 2θ of about 8.7°±0.3°. In another embodiment, the XRD pattern for Form II differs from the XRD pattern from Form I and includes a peak at 2θ of about 9.6°±0.3°. In another embodiment, the XRD pattern for Form II differs from the XRD pattern from Form I and includes peaks at 2θ of about 8.7°±0.3° and 9.6°±0.3°. Desirably, the XRD for Form II lacks a peak at 2θ of about 6.0°.

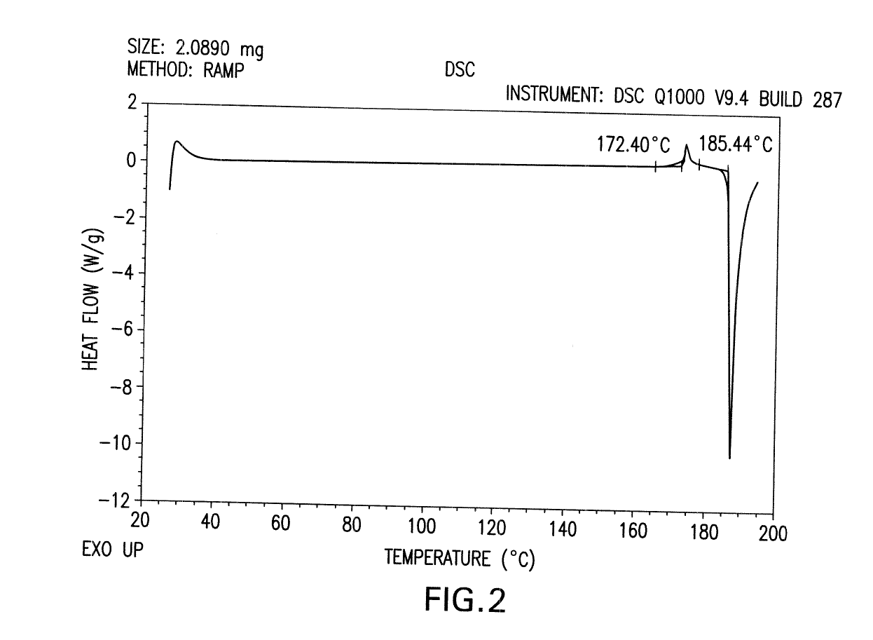

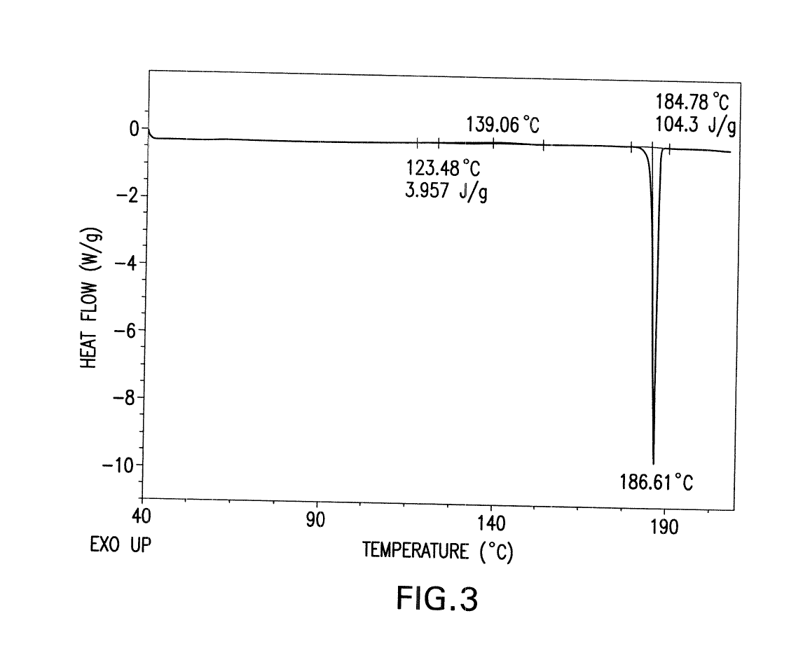

Polymorph Form II of 4-(2-(4,4-dimethyl-2-oxooxazolidin-3-yl)thiazol-4-yl)benzonitrile can be further characterized and distinguished from Form I by differential scanning calorimetry (DSC). DSC thermograms for Form II are provided in FIGS. 2 and 3, and were obtained using DSC techniques known to those of skill in the art. One of skill in the art would readily be able to determine the conditions necessary to obtain a DSC thermogram of Form II. A variety of differential scanning calorimeters are available to those of skill in the art and include the Q series™ DSC Q1000 (TA instruments) using temperatures of about 25° C. to about 220° C. and temperature increases at various rates including 5° C./minute, 10° C./minute, 20° C./minute, 30° C./minute, and 50° C./minute, among other instruments and conditions. One skilled in the art would recognize that the peak positions in the DSC thermogram can vary depending upon kinetic factors such as, for example, heating rate and particle size.

In one embodiment, the DSC thermogram of Form II differs from the DSC thermogram of Form I and includes an exothermic peak with a Tₒₙₛₑₜ of about 120 to about 160±1° C. and an endothermic peak with a Tₒₙₛₑₜ of about 185° C. In accordance with some embodiments, the DSC thermogram of Form II differs from the DSC thermogram of Form I and includes an exothermic peak with a Tₒₙₛₑₜ of about 160 to about 175±1° C. In other embodiments, the DSC thermogram for Form II differs from the DSC thermogram of Form I and includes an exothermic peak with a Tₒₙₛₑₜ of about 120 to about 130±1° C. The DSC thermogram of Form II can exhibit an exothermic peak due to thermal conversion of Form II to Form I.

Solid state nuclear magnetic resonance (SSNMR) spectroscopy can be utilized to distinguish polymorph Form II of 4-(2-(4,4-dimethyl-2-oxooxazolidin-3-yl)thiazol-4-yl)benzonitrile from Form I. One of skill in the art would readily be able to determine the conditions necessary to obtain a NMR spectrum of Form II. A variety of NMR instruments useful for solid state NMR is available and could readily be selected by those of skill in the art. Chemical shifts in solid state NMR are influenced by molecular packing and other solid-state effects, and differ for polymorphs with different crystal structures. Solid state NMR may be utilized for the analysis of both pure compounds and compounds present in pharmaceutical formulations (Munson and Lubach, Encyclopedia of Pharmaceutical Technology, 2006, 1:1, 3297-3310).

Solution phase NMR spectroscopy can be used to verify the purity and chemical structure of Form II 4-(2-(4,4-dimethyl-2-oxooxazolidin-3-yl)thiazol-4-yl)benzonitrile. A variety of NMR instruments for solution NMR is available and could readily be selected by those of skill in the art. One of skill in the art would also be able to readily select a suitable solvent, including isotope labeled solvents such as ²H or ¹³C labeled solvents. Those skilled in the art recognize that resolution and chemical shift is affected by the field strength of the NMR instrument and the choice of NMR solvent.

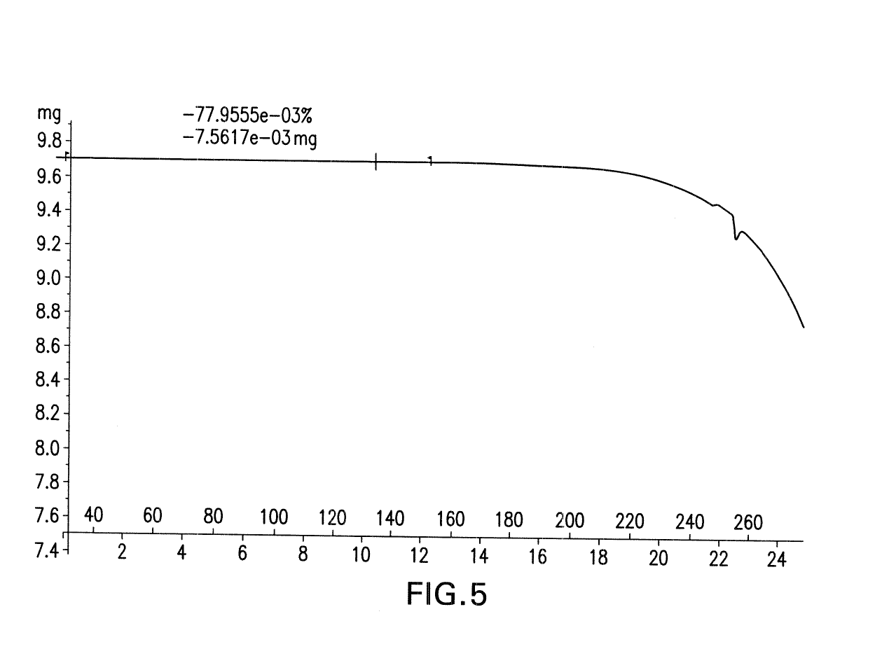

Thermogravimetric analysis (TGA) can be utilized to verify that a polymorph is anhydrous. In reference to FIG. 5, TGA indicated no weight loss for Form II; Form II is thus anhydrous.

Polymorph Form II of 4-(2-(4,4-dimethyl-2-oxooxazolidin-3-yl)thiazol-4-yl)benzonitrile can be micronized under nitrogen and conventional micronizing techniques, for example with a Trost or jet mill, as discussed above for Form I. Preferably, micronized 4-(2-(4,4-dimethyl-2-oxooxazolidin-3-yl)thiazol-4-yl)benzonitrile has a median particle size less than about 6.4 μm.

In accordance with one embodiment, polymorph Form II of 4-(2-(4,4-dimethyl-2-oxooxazolidin-3-yl)thiazol-4-yl)benzonitrile is preferably prepared from polymorph Form I of 4-(2-(4,4-dimethyl-2-oxooxazolidin-3-yl)thiazol-4-yl)benzonitrile. More desirably, polymorph Form II is prepared by dissolving polymorph Form I in methanol at an elevated temperature (i.e., a temperature above ambient). Desirably, the methanol solution is mixed to obtain a homogenous mixture. Preferably, the methanol solution is added to water and polymorph Form II of 4-(2-(4,4-dimethyl-2-oxooxazolidin-3-yl)thiazol-4-yl)benzonitrile crystallized. Isolation of Form II can be accomplished using techniques known to those of skill in the art including filtration, among others. In one aspect, the elevated temperature is from about 50 to about 60° C. In another aspect, the methanol solution is maintained at a temperature of about 60° C. In another aspect, the methanol solution is heated at reflux. In accordance with some embodiments, about equal volumes of methanol solution and water are combined. The water is preferably at ambient temperature before the addition of the methanol solution.

Embodiments of the present invention further provide processes for preparing polymorph Form I of 4-(2-(4,4-dimethyl-2-oxooxazolidin-3-yl)thiazol-4-yl)benzonitrile from polymorph Form II. In one embodiment, Form II is converted to Form I by slurrying polymorph Form II of 4-(2-(4,4-dimethyl-2-oxooxazolidin-3-yl)thiazol-4-yl)benzonitrile in a solvent such as methanol, water, a mixture comprising acetone and water, ethyl acetate, or acetone. Polymorph Form II of 4-(2-(4,4-dimethyl-2-oxooxazolidin-3-yl)thiazol-4-yl)benzonitrile is then isolated using techniques known to those of skill in the art. In one example, Form I is isolated by filtration after slurrying for about 7 days in a mixture of acetone and water in a 9:1 ratio.

In another embodiment, Form II is converted to Form I by heating polymorph Form II of 4-(2-(4,4-dimethyl-2-oxooxazolidin-3-yl)thiazol-4-yl)benzonitrile to about 160 to about 170° C.

The crystalline form of 4-(2-(4,4-dimethyl-2-oxooxazolidin-3-yl)thiazol-4-yl)benzonitrile may be prepared substantially as a single polymorph, i.e., greater than 95% of Form II, or may crystallize in combination with Form I or other polymorphs. In some embodiments, the crystalline form of 4-(2-(4,4-dimethyl-2-oxooxazolidin-3-yl)thiazol-4-yl)benzonitrile comprises at least 50% Form II. In some embodiments, In some embodiments, the crystalline form of 4-(2-(4,4-dimethyl-2-oxooxazolidin-3-yl)thiazol-4-yl)benzonitrile comprises at least 75% Form II. In still other embodiments, the crystalline form of 4-(2-(4,4-dimethyl-2-oxooxazolidin-3-yl)thiazol-4-yl)benzonitrile comprises at least 90% Form II.

The compound 4-(2-(4,4-dimethyl-2-oxooxazolidin-3-yl)thiazol-4-yl)benzonitrile has shown activity for modulating PR activity in assays to identify progestins or antiprogestins by (a) determining effect on alkaline phosphatase activity in T47D cells or (b) evaluating the progesterone receptor (PR) binding activity in live, intact (whole) cells using the human breast carcinoma T47D cell line and ³H-progesterone as the labeled ligand (data presented in Table below).

TABLE 2

| T47D Alkaline | PR T47D Whole |
|---|---|
| Phosphatase | Cell Binding, |
| Activity, IC₅₀ (nM) | Ki (nM) |
| 3.1 | 4.9 |

The compound 4-(2-(4,4-dimethyl-2-oxooxazolidin-3-yl)thiazol-4-yl)benzonitrile is useful for treating or preventing a condition modulated by progesterone and the progesterone receptor comprising the step of administering to said subject an effective amount of the compound as disclosed in U.S. Provisional Patent Application No. 60/837,898, filed Aug. 28, 2006, and U.S. patent application Ser. No. 11/891,748, filed on Aug. 13, 2007.

Form II is therefore useful in contraception and hormone replacement therapy. Form II is also useful in the treatment and/or prevention of fibroids, specifically uterine fibroids; benign prostatic hypertrophy; benign and malignant neoplastic disease; dysfunctional bleeding; uterine leiomyomata; endometriosis; polycystic ovary syndrome; and hormone-dependent carcinomas and adenocarcinomas of the pituitary, endometrium, kidney, uterine, ovary, breast, colon, and prostate and other hormone-dependent tumors. Form II is also useful for the synchronization of estrus. Additional uses of Form II include the stimulation of food intake. In one embodiment, the neoplastic disease is hormone-dependent.

In some embodiments, Form II of 4-(2-(4,4-dimethyl-2-oxooxazolidin-3-yl)thiazol-4-yl)benzonitrile can be administered in combination with other agents, such as, without limitation, progestins, antiprogestins, estrogens, antiestrogens, selective estrogen receptor modulators (SERMS), among others. Progestins can include, without limitation, tanaproget, levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, (17-deacetyl)norgestimate. Estrogens can include, without limitation, ethinyl estradiol. The compounds described herein can be combined with one or more of these agents, delivered concurrently therewith one or more of these agents, delivered prior to one or more of these agents, or delivered subsequent to one or more of these agents. In particular, it is contemplated that when Form II is used for contraception or hormone replacement therapy, it can be administered in conjunction with one or more other progesterone receptor agonists, estrogen receptor agonists, progesterone receptor antagonists, and selective estrogen receptor modulators, among others.

When utilized for treating neoplastic disease, carcinomas, and adenocarcinomas, Form II can be administered in conjunction with one or more chemotherapeutic agents which can readily be selected by one of skill in the art.

The present teachings provide pharmaceutical compositions comprising Form II of 4-(2-(4,4-dimethyl-2-oxooxazolidin-3-yl)thiazol-4-yl)benzonitrile alone or in combination with Form I or other polymorphs. In one embodiment, a pharmaceutical composition containing polymorph Form II of 4-(2-(4,4-dimethyl-2-oxooxazolidin-3-yl)thiazol-4-yl)benzonitrile and a pharmaceutically acceptable carrier is provided.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

The compositions typically contain a pharmaceutically acceptable carrier, but can also contain other suitable components. Typically, the additional components are inert and do not interfere with the function of the required components of the compositions. The compositions can further include other adjuvants, syrups, elixirs, diluents, binders, lubricants, surfactants, granulating agents, disintegrating agents, emollients, metal chelators, pH adjustors, surfactants, fillers, disintegrants, and combinations thereof, among others.

Adjuvants can include, without limitation, flavoring agents, coloring agents, preservatives, and supplemental antioxidants, which can include vitamin E, ascorbic acid, butylated hydroxytoluene (BHT) and butylated hydroxyanisole (BHA).

Binders can include, without limitation, povidone, cellulose, methylcellulose, hydroxymethylcellulose, carboxymethylcellulose calcium, carboxymethylcellulose sodium, hydroxypropylcellulose, hydroxypropylmethylcellulose phthalate, noncrystalline cellulose, polypropylpyrrolidone, polyvinylpyrrolidone (povidone, PVP), gelatin, gum arabic and acacia, polyethylene glycols, starch, sugars such as sucrose, kaolin, dextrose, and lactose, cholesterol, tragacanth, stearic acid, gelatin, casein, lecithin (phosphatides), cetostearyl alcohol, cetyl alcohol, cetyl esters wax, dextrates, dextrin, glyceryl monooleate, glyceryl monostearate, glyceryl palmitostearate, polyoxyethylene alkyl ethers, polyoxyethylene castor oil derivatives, polyoxyethylene stearates, polyvinyl alcohol, and gelatin, among others. In one embodiment, the binder is povidone.

Lubricants can include light anhydrous silicic acid, talc, stearic acid, sodium lauryl sulfate, magnesium stearate and sodium stearyl fumarate, among others. In one embodiment, the lubricant is magnesium stearate.

Granulating agents can include, without limitation, silicon dioxide, starch, calcium carbonate, pectin, crospovidone, and polyplasdone, among others.

Disintegrating agents or disintegrants can include starch, carboxymethylcellulose, substituted hydroxypropylcellulose, sodium bicarbonate, calcium phosphate, calcium citrate, sodium starch glycolate, pregelatinized starch or crospovidone, among others.

Emollients include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Surfactants include polysorbates, sorbitan esters, poloxamer, or sodium lauryl sulfate. In one embodiment, the surfactant is sodium lauryl sulfate.

Metal chelators include physiologically acceptable chelating agents including edetic acid, malic acid, or fumaric acid. In one embodiment, the metal chelator is edetic acid.

pH adjusters can be utilized to adjust the pH of a solution containing polymorph Form II of 4-(2-(4,4-dimethyl-2-oxooxazolidin-3-yl)thiazol-4-yl)benzonitrile to about 4, about 5, or about 6. In one embodiment, the pH of a solution containing polymorph Form II of 4-(2-(4,4-dimethyl-2-oxooxazolidin-3-yl)thiazol-4-yl)benzonitrile is adjusted to a pH of about 4.6. pH adjustors include physiologically acceptable agents including citric acid, ascorbic acid, fumaric acid, or malic acid, and salts thereof. In one embodiment, the pH adjuster is citric acid.

Additional fillers that can be used in the composition include mannitol, calcium phosphate, pregelatinized starch, or sucrose.

In one embodiment, a method of preparing a pharmaceutical composition containing polymorph Form II of 4-(2-(4,4-dimethyl-2-oxooxazolidin-3-yl)thiazol-4-yl)benzonitrile is described and includes combining polymorph Form II of 4-(2-(4,4-dimethyl-2-oxooxazolidin-3-yl)thiazol-4-yl)benzonitrile and one or more of a metal chelator, a pH adjuster, a surfactant, at least one filler, a binder, a disintegrant, and a lubricant.

The present teachings further provide methods of delivering polymorph Form II of 4-(2-(4,4-dimethyl-2-oxooxazolidin-3-yl)thiazol-4-yl)benzonitrile to a patient, where the method includes administering Form II. A patient or subject being treated is a mammalian subject and typically a female. Desirably, the subject is a human. However, as used herein, a female can include non-human mammals, e.g., cattle or livestock, horses, pigs, domestic animals, and others.

Polymorph Form II of 4-(2-(4,4-dimethyl-2-oxooxazolidin-3-yl)thiazol-4-yl)benzonitrile can be formulated in any form suitable for the desired route of delivery using a pharmaceutically effective amount of Form II. For example, Form II can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release. Desirably, delivery is oral.

For example, Form II may be formulated for administration orally in such forms as tablets, capsules, microcapsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like. The preferred pharmaceutical compositions from the standpoint of ease of preparation and administration are solid compositions, particularly tablets and hard-filled or liquid-filled capsules.

Form II may also be administered parenterally or intraperitoneally. Solutions or suspensions of Form II can be prepared in water suitably mixed with a surfactant such as hydroxypropylcellulose. Dispersions can also be prepared in glycerol, liquid, polyethylene glycols and mixtures thereof in oils. Under ordinary conditions of storage and use, these preparations contain a preservative to prevent the growth of microorganisms. Typically, such sterile injectable solutions or suspensions contain from about 0.05 to 5% suspending agent in an isotonic medium. Such pharmaceutical preparations may contain, for example, from about 25 to about 90% of the active ingredient in combination with the carrier, more usually between about 5% and 60% by weight.

In another embodiment, Form II is delivered intravenously, intramuscularly, subcutaneously, parenterally and intraperitoneally in the form of sterile injectable solutions, suspensions, dispersions, and powders which are fluid to the extent that easy syringe ability exits. Such injectable compositions are sterile, stable under conditions of manufacture and storage, and free of the contaminating action of microorganisms such as bacteria and fungi.

The carrier can be a solvent or dispersion medium containing, for example, water, ethanol, polyol (e.g., glycerol, propylene glycol and liquid polyethylene glycol), oils, and mixtures thereof. Desirably the liquid carrier is water. In one embodiment, the oil is vegetable oil. Optionally, the liquid carrier contains a suspending agent. In another embodiment, the liquid carrier is an isotonic medium and contains 0.05 to about 5% suspending agent.

In another embodiment, Form II is delivered rectally in the form of a conventional suppository.

In another embodiment, Form II is delivered vaginally in the form of a conventional suppository, cream, gel, ring, or coated intrauterine device (IUD).

In another embodiment, Form II is delivered intranasally or intrabronchially in the form of an aerosol.

In another embodiment, Form II is delivered transdermally or by sustained release through the use of a transdermal patch containing Form II and an optional carrier that is inert to Form II, is nontoxic to the skin, and allows for delivery of Form II for systemic absorption into the blood stream. Such a carrier can be a cream, ointment, paste, gel, or occlusive device. The creams and ointments can be viscous liquid or semisolid emulsions. Pastes include absorptive powders dispersed in petroleum or hydrophilic petroleum. Further, a variety of occlusive devices can be utilized to release Form II into the blood stream and include semi-permeable membranes covering a reservoir contain the active reagents, or a matrix containing the reactive reagents.

The use of sustained delivery devices can be desirable, in order to avoid the necessity for the patient to take medications on a daily basis. The term “sustained delivery” is used herein to refer to delaying the release of an active agent, such as polymorph Form II of 4-(2-(4,4-dimethyl-2-oxooxazolidin-3-yl)thiazol-4-yl)benzonitrile, until after placement in a delivery environment, followed by a sustained release of the agent at a later time. A number of sustained delivery devices are known in the art and include, without intended limitation, hydrogels (U.S. Pat. Nos. 5,266,325; 4,959,217; 5,292,515, among others), osmotic pumps (U.S. Pat. Nos. 4,295,987 and 5,273,752 and European Patent No. 314,206, among others); hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devices composed of, for example, polyesters, polyanhydrides, or lactic acid/glycolic acid copolymers (U.S. Pat. No. 5,817,343). Additional methods and devices for drug delivery are recognized in the art as in, for example, U.S. Pat. Nos. 3,845,770; 3,916,899; 3,536,809; 3,598,123; and 4,008,719. For use in such sustained delivery devices, Form II can be formulated as described herein.

Desirably, Form II is formed into a suitable dosing unit for delivery to a patient. Suitable dosing units include oral dosing units, such as a directly compressible tablets, capsules, powders, suspensions, microcapsules, dispersible powders, granules, suspensions, syrups, elixirs, and aerosols. In one embodiment, Form II is compressed into a tablet, which is optionally added to a capsule, or Form II is added directly to a capsule. Form II can also be formulated for delivery by other suitable routes. These dosing units are readily prepared using the methods described herein and those known to those of skill in the art.

Solid forms, including tablets, caplets, and capsules containing polymorph Form II of 4-(2-(4,4-dimethyl-2-oxooxazolidin-3-yl)thiazol-4-yl)benzonitrile can be formed by dry blending polymorph Form II of 4-(2-(4,4-dimethyl-2-oxooxazolidin-3-yl)thiazol-4-yl)benzonitrile with the components described above. In one embodiment, the capsules utilized include hydroxypropyl methylcellulose, hypromellose capsule, or a hard shell gelatin capsule. The tablets or caplets that contain polymorph Form II of 4-(2-(4,4-dimethyl-2-oxooxazolidin-3-yl)thiazol-4-yl)benzonitrile are optionally film-coated. Suitable film-coatings are known to those of skill in the art. For example, the film-coating can be selected from among polymers such as hydroxypropylmethylcellulose, ethyl cellulose, polyvinyl alcohol, and combinations thereof.

A pharmaceutically effective amount of Form II can vary depending on the other components of the composition being delivered, mode of delivery, severity of the condition being treated, the patient's agent and weight, and any other active ingredients used in the composition. The dosing regimen can also be adjusted to provide the optimal therapeutic response. Several divided doses can be delivered daily, e.g., in divided doses 2 to 4 times a day, or a single dose can be delivered. The dose can however be proportionally reduced or increased as indicated by the exigencies of the therapeutic situation. In one embodiment, the delivery is on a daily, weekly, or monthly basis. In another embodiment, the delivery is on a daily delivery. However, daily dosages can be lowered or raised based on the periodic delivery.

The dosage requirements of Form II may vary based on the severity of the symptoms presented and the particular subject being treated. Treatment can be initiated with small dosages less than the optimum dose of Form II. Thereafter the dosage is increased until the optimum effect under the circumstances is reached. Precise dosages will be determined by the administering physician based on experience with the individual subject treated. In general, Form II is most desirably administered at a concentration that will generally afford effective results without causing any unacceptable harmful or deleterious side effects. For example, an effective amount of Form II is generally about 0.05 mg to about 1 mg, about 0.05 mg to about 0.3 mg, about 0.05 mg, about 0.075 mg, about 0.1 mg, about 0.15 mg, about 0.2 mg, or about 0.3 mg.

Also provided in accordance with embodiments of the invention herein are kits or packages containing polymorph Form II of 4-(2-(4,4-dimethyl-2-oxooxazolidin-3-yl)thiazol-4-yl)benzonitrile. Kits can include Form II, alone or in combination with Form I or other polymorphs, and a carrier suitable for administration to a mammalian subject as discussed above. Typically, the tablets or capsules are packaged in blister packs, and desirably 2.00 mil polychlorotrifluorethylene (PCTFE) polymer, such as Ultrx™ 2000, blister packs. In one embodiment, a kit is provided and contains polymorph Form II of 4-(2-(4,4-dimethyl-2-oxooxazolidin-3-yl)thiazol-4-yl)benzonitrile; and a carrier suitable for administration to a mammalian subject is described.

The kits or packages containing Form II are designed for use in the regimens described herein. These kits are desirably designed for daily oral delivery over 21-day, 28-day, 30-day, or 31-day cycles, among others, and more desirably for one oral delivery per day. When Form II is to be delivered continuously, a package or kit can include Form II in each tablet. When Form II is to be delivered with periodic discontinuation, a package or kit can include placebos on those days when Form II is not delivered.

Additional components may be co-administered with Form II and include progestational agents, estrogens, and selective estrogen receptor modulators.

The kits are also desirably organized to indicate a single oral formulation or combination of oral formulations to be taken on each day of the cycle, desirably including oral tablets to be taken on each of the days specified, and more desirably one oral tablet will contain each of the combined daily dosages indicated.

In one embodiment, a kit can include a single phase of a daily dosage of Form II over a 21-day, 28-day, 30-day, or 31-day cycle. Alternatively, a kit can include a single phase of a daily dosage of Form II over the first 21 days of a 28-day, 30-day, or 31-day cycle. A kit can also include a single phase of a daily dosage of Form II over the first 28 days of a 30-day or 31-day cycle.

In another embodiment, a kit can include a single combined phase of a daily dosage of Form II and a progestational agent over a 21-day, 28-day, 30-day, or 31-day cycle. Alternatively, a kit can include a single combined phase of a daily dosage of Form II and a progestational agent over the first 21 days of a 28-day, 30-day, or 31-day cycle. A kit can also include a single combined phase of a daily dosage of Form II and a progestational agent over the first 28 days of a 30-day or 31-day cycle.

In another embodiment, a 28-day kit can include a first phase of from 14 to 28 daily dosage units of Form II; a second phase of from 1 to 11 daily dosage units of a progestational agent; and, optionally, a third phase of an orally and pharmaceutically acceptable placebo for the remaining days of the cycle.

In yet a further embodiment, a 28-day kit can include a first phase of from 14 to 21 daily dosage units of Form II; a second phase of from 1 to 11 daily dosage units of a progestational agent; and, optionally, a third phase of an orally and pharmaceutically acceptable placebo for the remaining days of the cycle.

In another embodiment, a 28-day kit can include a first phase of from 18 to 21 daily dosage units of Form II; a second phase of from 1 to 7 daily dose units of a progestational agent; and, optionally, an orally and pharmaceutically acceptable placebo for each of the remaining 0 to 9 days in the 28-day cycle.

In yet a further embodiment, a 28-day kit can include a first phase of 21 daily dosage units of Form II; a second phase of 3 daily dosage units for days 22 to 24 of a progestational agent; and, optionally, a third phase of 4 daily units of an orally and pharmaceutically acceptable placebo for each of days 25 to 28.

In another embodiment, a 28-day kit can include a first phase of from 14 to 21 daily dosage units of a progestational agent equal in progestational activity to about 35 to about 150 μg levonorgestrel, a second phase of from 1 to 11 daily dosage units of Form II; and optionally, a third phase of an orally and pharmaceutically acceptable placebo for the remaining days of the cycle in which no antiprogestin, progestin or estrogen is administered.

In another embodiment, a 28-day kit can include a first phase of from 14 to 21 daily dosage units of a progestational agent equal in progestational activity to about 35 to about 100 μg levonorgestrel; a second phase of from 1 to 11 daily dosage units of Form II; and optionally, a third phase of an orally and pharmaceutically acceptable placebo for the remaining days of the cycle in which no antiprogestin, progestin or estrogen is administered.

Desirably, the daily dosage of Form II remains fixed in each particular phase in which it is delivered. It is further preferable that the daily dose units described are to be delivered in the order described, with the first phase followed in order by the second and third phases. To help facilitate compliance with each regimen, it is also preferred that the kits contain the placebo described for the final days of the cycle.

A number of packages or kits are known in the art for the use in dispensing pharmaceutical agents for oral use. Desirably, the package has indicators for each day of the 28-day cycle, and more desirably is a labeled blister package, dial dispenser package, or bottle.

The kit can further contain instructions for administering Form II.

Variations, modifications, and other implementations of what is described herein will occur to those of ordinary skill in the art without departing from the spirit and the essential characteristics of the present teachings.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### EXAMPLES

Differential scanning calorimetry data were collected using a Q series™ DSC Q1000 (TA instruments) under the following parameters:

| | |
|---|---|
| purge gas (N₂): | 50 mL/minute; |
| scan range:: | 40 to 200° C.; |
| scan rate: | 10° C./minute. |

Thermogravimetric analysis (TGA) data was collected using a TGA/SDTA 851e instrument (Mettler Toledo) under the following parameters:

| | |
|---|---|
| purge gas (N₂): | 40 mL/minute; |
| scan range: | 30 to 250° C.; |
| scan rate: | 10° C./minute. |

X-Ray diffraction data was acquired using a D8 ADVANCE X-ray powder diffractometer (Bruker) having the following parameters:

| | |
|---|---|
| voltage: | 40 kV; |
| current: | 40.0 mA; |
| scan range (2θ): | 5 to 30°; |
| scan step size: | 0.01°; |
| total scan time: | 20 minutes; |
| detector: | VANTEC; and |
| antiscattering slit: | 1 mm. |

### Example 1

### PREPARATION OF POLYMORPH FORM II OF 4-(2-(4,4-DIMETHYL-2-OXOOXAZOLIDIN-3-YL)THIAZOL-4-YL)BENZONITRILE

Polymorph Form II of 4-(2-(4,4-dimethyl-2-oxooxazolidin-3-yl)thiazol-4-yl)benzonitrile was prepared by dissolving polymorph Form I of 4-(2-(4,4-dimethyl-2-oxooxazolidin-3-yl)thiazol-4-yl)benzonitrile in 80 volumes of methanol at reflux. The methanol solution was then added to an equal volume of water and Form II immediately precipitated from the solution. The crystals of Form II were collected by filtration and dried overnight at about 40-50° C. under a vacuum.

Form II of 4-(2-(4,4-dimethyl-2-oxooxazolidin-3-yl)thiazol-4-yl)benzonitrile thus obtained was analyzed using XRD, DSC, and NMR. The XRD pattern is provided in FIG. 1 and peak data is compiled in Table 1. FIG. 2 provides one DSC graph and displays one endothermic peak with a melting onset at about 185° C. and one exothermic peak with a melting onset at about 160 to about 170° C. FIG. 3 provides another DSC graph and displays one endothermic peak with a melting onset at about 185° C. and one exothermic peak with a melting onset at about 129° C. In reference to FIG. 5, the TGA data show no weight loss up to the melting temperatures, thereby verifying that Form II is anhydrous.

TABLE 1

| Angle | d value | Intensity |
|---|---|---|
| (2θ°) | (Å) | (%) |
| 8.67 | 10.19 | 80.70 |
| 9.70 | 9.11 | 48.40 |
| 12.29 | 7.20 | 13.50 |
| 13.10 | 6.75 | 4.80 |
| 15.55 | 5.69 | 1.70 |
| 17.37 | 5.10 | 96.00 |
| 17.92 | 4.95 | 33.50 |
| 21.78 | 4.08 | 0.50 |
| 23.39 | 3.80 | 5.50 |
| 25.89 | 3.44 | 7.50 |
| 26.17 | 3.40 | 100.00 |
| 27.70 | 3.22 | 1.70 |
| 27.99 | 3.19 | 1.10 |
| 28.74 | 3.10 | 0.70 |

The proton NMR spectrum of Form II was obtained in dimethylsulfoxide (DMSO-d₆) to confirm the identity and purity of 4-(2-(4,4-dimethyl-2-oxooxazolidin-3-yl)thiazol-4-yl)benzonitrile. ¹H NMR (DMSO-d₆) 1.77 (s, 6H), 4.36 (s, 2H), 7.91 (d, 2H), 8.04 (s, 1H), 8.11 (d, 2H).

### Example 2

### SOLUBILITY OF POLYMORPH FORM II OF 4-(2-(4,4-DIMETHYL-2-OXOOXAZOLIDIN-3-YL)THIAZOL-4-YL)BENZONITRILE IN VARIOUS SOLVENTS

In this example, the solubility of polymorph Form II of 4-(2-(4,4-dimethyl-2-oxooxazolidin-3-yl)thiazol-4-yl)benzonitrile in water is measured.

It is expected that polymorph form II of 4-(2-(4,4-dimethyl-2-oxooxazolidin-3-yl)thiazol-4-yl)benzonitrile will be more soluble in water than the corresponding polymorph Form I of 4-(2-(4,4-dimethyl-2-oxooxazolidin-3-yl)thiazol-4-yl)benzonitrile.

### Example 3

### CONVERTING POLYMORPH FORM II OF 4-(2-(4,4-DIMETHYL-2-OXOOXAZOLIDIN-3-YL)THIAZOL-4-YL)BENZONITRILE TO FORM I

A. Method One. Polymorph Form I of 4-(2-(4,4-dimethyl-2-oxooxazolidin-3-yl)thiazol-4-yl)benzonitrile was prepared from polymorph Form II by slurrying excess polymorph Form II of 4-(2-(4,4-dimethyl-2-oxooxazolidin-3-yl)thiazol-4-yl)benzonitrile in methanol, water, a mixture of acetone and water at a 9:1 ratio, ethyl acetate, or acetone for 7 days and collecting polymorph Form I of 4-(2-(4,4-dimethyl-2-oxooxazolidin-3-yl)thiazol-4-yl)benzonitrile using filtration, following by drying overnight at about 40-50° C. under a vacuum.

B. Method Two. Polymorph Form I of 4-(2-(4,4-dimethyl-2-oxooxazolidin-3-yl)thiazol-4-yl)benzonitrile was prepared from polymorph Form II by heating polymorph Form II of 4-(2-(4,4-dimethyl-2-oxooxazolidin-3-yl)thiazol-4-yl)benzonitrile to about 160° C. in a crucible. Form I was then collected from the crucible.

All publications cited in this specification are incorporated herein by reference. While the invention has been described with reference to a particularly preferred embodiment, it will be appreciated that modifications can be made without departing from the spirit of the invention. Such modifications are intended to fall within the scope of the appended claims. Accordingly, the scope of the invention is not to be considered as being limited by the preceding illustrative description but instead by the following claims, and all changes that come within the meaning and range of equivalency of the claims are intended to be embraced therein.

What is claimed is:

1. A crystalline form of 4-(2-(4,4-dimethyl-2-oxooxazolidin-3-yl)thiazol-4-yl)benzonitrile characterized by an X-ray diffraction comprising peaks at the following angles (±0.3°) of 2θ in its X-ray diffraction pattern: 8.67 and 9.70.

2. The crystalline form of 4-(2-(4,4-dimethyl-2-oxooxazolidin-3-yl)thiazol-4-yl)benzonitrile of claim 1, wherein said X-ray diffraction further comprises peaks at the following angles (±0.3°) of 2θ in its X-ray diffraction pattern: 17.37, 17.92, and 26.12.

3. The crystalline form of 4-(2-(4,4-dimethyl-2-oxooxazolidin-3-yl)thiazol-4-yl)benzonitrile of claim 1, wherein said X-ray diffraction further comprises peaks at the following angles (±0.3°) of 2θ in its X-ray diffraction pattern: 12.29, 13.10, 17.37, 17.92, 23.39, 25.81, and 26.12 degrees 2θ.

4. The crystalline form of 4-(2-(4,4-dimethyl-2-oxooxazolidin-3-yl)thiazol-4-yl)benzonitrile of claim 1 comprising at least 75% Form II.

5. The crystalline form of 4-(2-(4,4-dimethyl-2-oxooxazolidin-3-yl)thiazol-4-yl)benzonitrile of claim 5 comprising at least 90% Form II.

6. The crystalline form of 4-(2-(4,4-dimethyl-2-oxooxazolidin-3-yl)thiazol-4-yl)benzonitrile of claim 1, characterized by a differential scanning calorimetry thermogram having an endothermic peak with a Tₒₙₛₑₜ of about 185° C.

7. The crystalline form of 4-(2-(4,4-dimethyl-2-oxooxazolidin-3-yl)thiazol-4-yl)benzonitrile of claim 6 wherein said differential scanning calorimetry thermogram further has an exothermic peak with a Tₒₙₛₑₜ of about 120 to about 175° C.

8. The crystalline form of 4-(2-(4,4-dimethyl-2-oxooxazolidin-3-yl)thiazol-4-yl)benzonitrile of claim 7 wherein said differential scanning calorimetry thermogram further has an exothermic peak with a Tₒₙₛₑₜ of about 160 to about 175° C.

9. The crystalline form of 4-(2-(4,4-dimethyl-2-oxooxazolidin-3-yl)thiazol-4-yl)benzonitrile of claim 7 wherein said differential scanning calorimetry thermogram further has an exothermic peak with a Tₒₙₛₑₜ of about 120 to about 130° C.

10. A method of preparing Form II of 4-(2-(4,4-dimethyl-2-oxooxazolidin-3-yl)thiazol-4-yl)benzonitrile comprising:
(a) dissolving polymorph Form I of 4-(2-(4,4-dimethyl-2-oxooxazolidin-3-yl)thiazol-4-yl)benzonitrile in methanol at an elevated temperature; and
(b) combining the solution formed in step (a) with water.

11. The method of claim 10, wherein the ratio of methanol to water is 1:1 (v/v).

12. The method of claim 10, wherein the methanol solution is added to water in step (b).

13. The method of claim 10, wherein the elevated temperature in step (a) is between about 50 to 60° C.

14. The crystalline form of 4-(2-(4,4-dimethyl-2-oxooxazolidin-3-yl)thiazol-4-yl)benzonitrile prepared according to the method of claim 10.

15. A micronized form of 4-(2-(4,4-dimethyl-2-oxooxazolidin-3-yl)thiazol-4-yl)benzonitrile which has a median particle size less than about 6.4 μm.

16. A pharmaceutical composition comprising the crystalline form of 4-(2-(4,4-dimethyl-2-oxooxazolidin-3-yl)thiazol-4-yl)benzonitrile of claim 1 and a pharmaceutically acceptable carrier.

17. A kit comprising the crystalline form of 4-(2-(4,4-dimethyl-2-oxooxazolidin-3-yl)thiazol-4-yl)benzonitrile of claim 1 and a carrier suitable for administration to a mammalian subject.

18. A method of contraception comprising administering to a female in need thereof a crystalline form of 4-(2-(4,4-dimethyl-2-oxooxazolidin-3-yl)thiazol-4-yl)benzonitrile of claim 1.

19. A method for treating or preventing fibroids comprising administering to a female in need thereof a crystalline form of 4-(2-(4,4-dimethyl-2-oxooxazolidin-3-yl)thiazol-4-yl)benzonitrile of claim 1.

20. The method of claim 19, wherein said fibroids are uterine fibroids.

21. A method for treating or preventing uterine leiomyomata comprising administering to a female in need thereof a crystalline form of 4-(2-(4,4-dimethyl-2-oxooxazolidin-3-yl)thiazol-4-yl)benzonitrile of claim 1.

22. A method for treating or preventing endometriosis, dysfunctional bleeding, and polycystic ovary syndrome comprising administering to a female in need thereof a crystalline form of 4-(2-(4,4-dimethyl-2-oxooxazolidin-3-yl)thiazol-4-yl)benzonitrile of claim 1.

23. A method for treating or preventing hormone-dependent carcinomas comprising administering to mammal in need thereof a crystalline form of 4-(2-(4,4-dimethyl-2-oxooxazolidin-3-yl)thiazol-4-yl)benzonitrile of claim 1.

24. The method of claim 23, wherein said carcinomas are selected from the group consisting of carcinomas of the endometrium, breast, uterine, ovarian and prostate cancer.

25. A method of providing hormone replacement therapy comprising administering to a female in need thereof a crystalline form of 4-(2-(4,4-dimethyl-2-oxooxazolidin-3-yl)thiazol-4-yl)benzonitrile of claim 1.

26. A method of stimulating food intake comprising administering to a mammal in need thereof a crystalline form of 4-(2-(4,4-dimethyl-2-oxooxazolidin-3-yl)thiazol-4-yl)benzonitrile of claim 1.

27. A method of synchronizing estrus comprising administering to a mammal in need thereof a crystalline form of 4-(2-(4,4-dimethyl-2-oxooxazolidin-3-yl)thiazol-4-yl)benzonitrile of claim 1.

28. A method for preparing Form I of 4-(2-(4,4-dimethyl-2-oxooxazolidin-3-yl)thiazol-4-yl)benzonitrile comprising slurrying polymorph Form II of 4-(2-(4,4-dimethyl-2-oxooxazolidin-3-yl)thiazol-4-yl)benzonitrile for 7 days in a solvent comprising methanol, water, a mixture comprising acetone and water at a 9:1 ratio, ethyl acetate, or acetone.

29. A method for preparing Form I of 4(2-(4,4-dimethyl-2-oxooxazolidin-3-yl)thiazol-4-yl)benzonitrile comprising heating polymorph Form II of 4-(2-(4,4-dimethyl-2-oxooxazolidin-3-yl)thiazol-4-yl)benzonitrile to about 160 to about 170° C.
